(19) United States
(12) Patent Application Publication
(10) Pub. No.: US 2022/0175974 A1
(43) Pub. Date: Jun. 9, 2022

(54) TARGETING COMPOUNDS FOR CANCERS SELECTED FROM ESOPHAGUS, PHARYNX AND LARYNX, LUNG, BRAIN, AND INTESTINES

(71) Applicant: HQ MEDICAL NETHERLANDS B.V., AC Rotterdam (NL)

(72) Inventors: Markwin Hendrik Maring, AC ROTTERDAM (NL); Carel Jan Vogels, AC ROTTERDAM (NL)

(73) Assignee: HQ MEDICAL NETHERLANDS B.V., AC Rotterdam (NL)

(21) Appl. No.: 17/599,379

(22) PCT Filed: Mar. 27, 2020
(86) PCT No.: PCT/NL2020/050213

§ 371 Date: Sep. 28, 2021

(30) Foreign Application Priority Data
Mar. 28, 2019 (NL) 2022828

(51) Int. Cl. A61K 51/04; A61K 41/00; A61P 35/00

(52) U.S. Cl. CPC A61K 51/0497; A61P 35/00; A61K 41/0052; A61K 51/0446

(57) ABSTRACT

Systems for use as a medicament for cancers selected from esophagus, pharynx and larynx, lung, brain, and intestines, include a targeting molecule for binding necrotic cells, a chelator, and a radionuclide, to a dosage comprising the system. In particular, the systems relate to compositions targeting necrotic cells.

### FIELD OF THE INVENTION

The invention relates to a system for use as a medicament for cancers selected from esophagus, pharynx and larynx, lung, brain, and intestines, comprising a targeting molecule for binding necrotic cells, a chelator, and a radionuclide, to a dosage comprising the system. In particular the invention relates to compositions targeting necrotic cells.

### BACKGROUND OF THE INVENTION

The present inventions relates to targeting of necrotic cells with cyanines. Targeting of necrosis is specific and unique because necrosis only appears in pathological conditions of cell death due to an insufficient blood supply and thus a lack of oxygen, trauma or due to direct cytotoxic agents or other cancer treatments like radiotherapy or photo dynamic therapy. This is in contrast to apoptotic cell death, which occurs continuously during tissue turnover. Therefore apoptotic cell death, in contrast to necrotic cell death, is not useable for targeting diseases characterized by necrosis as found for instance in case of tumours (rapidly growing tumours spontaneously develop necrotic cores), trauma, infarcts, osteoarthritis, diabetes, arteriosclerotic plaques, burns, certain bacterial infections, etc. The present compounds specifically bind to intracellular proteins.

Further details on background may be obtained from previously filed applications WO 2013/119111 A1, WO 2013/119114 A1, and WO 2014/123418 A1, which documents and their contents are hereby incorporated by reference.

Further background art relates to the following documents. US 2016/263249 A1 recites near infrared fluorescent contrast bioimaging agents and methods of use thereof in the medical field, particularly in in vitro diagnostics, in vivo diagnostics, and image-guided surgery, such the use of certain compounds for labelling or likewise contrast agent. Such compounds may be conjugated. Such uses are widely known. Stammes et al., in various documents (Molecular Imaging & Biology, Vol. 18, No. 6, 2016-06-08, p. 905-915, Radiotherapy and Oncology, Vol. 111, May 2015, p. 5124-5125, and Frontiers in Oncology, Vol. 6, 2016-10-21, p. 1-11) recites cyanine based SPECT-probes for imaging, the use of a specific cyanine for detecting effectiveness of radiotherapy, and the use of a specific cyanine-conjugate for monitoring induced tumor cell death by radiation therapy. Fernandes et al. in Biomedicine & Pharmacotherapy, Vol. 95, November 2017, p. 469-476) recites the role of radionuclide probes for monitoring anti-tumor drugs efficacy in a review paper. Zhang et al. in Photochemistry and Photobiology, Vol. 81, November 2005, p. 1499-1504, recites spectral properties of imaging agents with a chelator metal complex, such as a chelator coupled to a radionuclide for PET or SPECT imaging, such as for diagnostic information. These articles are more concerned with imaging/monitoring than targeting or treating. Also the radionuclide probes are typically used as separate entities.

The present invention does not relate to DNA binding. DNA-binding molecules are generally considered unsuitable for use in humans due the high chance that such molecules are toxic and possibly also mutagenic/carcinogenic.

It is noted that all proteins comprise amines. Amine reactive molecules will bind to any protein without large differences in affinity. In biological samples, such as blood and tissue, there is a vast amount of extracellular protein present. Selectivity for e.g. dead cells in the sample cannot therefore be achieved. In vivo, strong covalent binding to proteins will result in only very slow clearance from the body, which increases the chance of adverse effects.

It is an object of the present invention to overcome one or more disadvantages of the compositions of the prior art and to provide alternatives to current compositions for diagnosis and treatment of cancers and other diseases involving necrotic cell death, without jeopardizing functionality and advantages.

### SUMMARY OF THE INVENTION

The present invention relates to a system comprising a targeting molecule for binding to necrotic cells, a chelator, and a radionuclide according to claim 1.

The present system comprises at least three entities, the entities being joined or linked, such as by a chemical or physical bond, each entity serving a distinct function within the system.

The present targeting molecule is very selective and very specific in binding to necrotic cells. It is non-activated, is capable of non-covalently binding to intracellular proteins when the membrane integrity of a cell is lost, and does not significantly interact with DNA. For the present invention four specific cyanines are selected, namely HQ4, HQ5, CW-800 and ZW-800. These molecules are found to have a high affinity towards the intracellular proteins, including tubulin and actin. In addition these cyanines are found to be cleared in the human body specifically well. As a result very low concentrations c.q. amounts of the present system may be used to provide advantageous effects thereof. It is noted that apoptosis specific probes, in contrast to necrosis specific probes will also target healthy tissue as apoptosis is involved in normal tissue turnover. The present cyanine is attached to a second entity, namely a chelator, wherein the chelator is selected from DOTA and NOTA (DOTA: 1,4,7,10-Tetraazacyclododecane-1,4,7,10-tetraacetic acid and NOTA: 1,4,7-triazacyclononane-N,N′,N″-triacetic acid). Attached to the chelator is attached a third entity, namely a radionuclide selected from Cu, In, Gd, Ga, Lu, Y, I, and Zr. The present system is found to be effective as a medicament, also referred to as drug, for cancers selected from esophagus, pharynx and larynx, lung, brain, and intestines. These cancers are considered to relate to and being associated with the Central Nervous system, the respiratory tract, the gastro-intestinal tract, the uro-genital tract, and head-neck region, respectively. These cancers are found difficult to treat, and have a relatively bad prognosis, and the present system can be used in low dosage and yet be very effective. The drug typically relates to a substance intended for use in the diagnosis, therapy, such as radio-therapy, hyperthermia therapy, and radio frequency ablation (RFA) therapy, cure, treatment, or prevention of the aforementioned diseases. For diagnosis relatively short-lived radionuclides (half-life between brackets) may be used, such as ⁶⁴Cu (12.7h), ⁶⁷Cu (61.8h), ⁶⁶Ga (9.5h), ⁶⁷Ga (3.3d), ⁶⁸Ga (1.2h), ⁷²Ga (14.1h), ⁷³Ga (4.9h), ⁸⁹Zr (78.4h), ⁸⁷Y (3.4d), ⁹⁰Y (2.7d), ¹¹¹In (2.8d), ¹²³I (13h), ¹²⁴I (4.2d), ¹³¹I (8.0d), ¹⁵⁹Gd (18.5h), and ¹⁷⁷Lu (6.6d), whereas for treatment relatively long-lived radionuclides may be used, such as ⁸⁸Zr (83.4d), ⁹⁵Zr (64.0d), ⁸⁸Y (106.6d), ⁹¹Y (58.5d), ¹¹⁴In (>>1y), ¹²⁵I (59.4d), ¹³¹I (8.0d), ¹⁵²Gd(>>1y), ¹⁵³Gd (240d), and ¹⁷³Lu (1.37y).

It has been found in earlier research by the present inventors that the targeting molecule specifically binds non-covalently to intracellular proteins such as actin, which are only available for the targeting molecule when the membrane integrity of a cell is lost, that is in case of a necrotic cell. For optimal targeting, the necrotic cells, are preferably in an early stage of necrosis, such as cells that have been dead for less than a few days, preferably less than half a day, such as a few hours, such as 2 hours, such as just dead cells. Characteristics of targeting molecules of the present invention, responsible for their effective targeting and/or safety, are that they are cell membrane impermeant, i.e. they cannot (significantly) cross the cell membrane of healthy cells; they are non-activated; they are capable of non-covalently binding to intracellular proteins (their target molecules); and, they are not capable/do not significantly bind to DNA (or RNA).

The present system relates to non-toxic small molecules, having an ability to bind to necrotic cells and tissue. These molecules do not interact with DNA, i.e. these are not toxic or mutagenic. Typically the present system has a wide-spread biodistribution, cross the blood-brain barrier, do not bind to cell-surface proteins and are sufficiently stable.

In an example crucial time is saved during cancer treatment. The present system instantly provides relevant data on the efficacy of the administered chemotherapeutic drug. An advantage of the present system is that it informs within 24-36 hours after a start of chemotherapy whether or not the therapy is effective. The use will prevent patients from enduring heavy treatments without clinical benefits, which is regarded a major improvement in cancer therapy. The use will result in cost savings. It is noted that unfortunately a (positive) prior art response rate to chemotherapy is limited to 20-35%. Further, the number of treatment cycles is typically limited to a maximum of four due to limitations of the human body. In other words it is crucial to identify a suitable therapy right from the start, before starting a cycle. By identifying at an early stage of treatment if a treatment is effective the treatment per se can be carried out if considered effective and can be skipped if considered not effective. In the case of a not effective treatment a second treatment can be started likewise. Such can be repeated a further number of times. Once a treatment is considered effective a cycle of chemotherapy can be initiated. It is noted that the identification can be repeated for every and any chemotherapy.

The present system also prevents over-treatment, saving on medicines used to prevent side-effects of cytotoxic drugs.

With the present system there is no need to culture harvested tumour cell e.g. in order to perform tests, which can take a relatively long period of time e.g. a week. The necrotic cells are marked in their natural environment, which e.g. reduces a risk of human errors.

Targeting molecules of the present invention have been found to bind to dead (necrotic) cells very selectively.

As mentioned earlier it has been found by the present inventors that necrotic cells and/or necrosis in general are attractive targets. Such relates to the observation that regions of necrotic cells are typically present in cancers (tumours), e.g. due to an insufficient blood supply and thus lack of oxygen, and in (or result from) diseases involving necrotic cell death. It is noted that regions of necrotic cells are not typically found inside of healthy tissue.

With regards to cancer, once a tumour has been identified, further necrotic cell death can be induced intentionally (for instance by local irradiation, photo dynamic or local thermal therapy and/or focused ultrasound) in part of the tumour to provide a larger target for the composition. Furthermore, wherein the present composition is used for therapeutic purposes, the number of necrotic cells will increase as the therapy progresses thus resulting in dose amplification as a function of time.

A useful discussion on the classification of cell death can be found in Kroemer et al, Cell Death and Differentiation, 2005, 12, 1463. In the present application, necrotic cells are taken as cells whose plasma membrane has lost integrity. A person of skill in the art is able to determine whether a plasma membrane is intact i.e. integral, such as through using fluorescent dyes, such as using commercial amine reactive dyes. The selectivity of the composition of the present invention for dead cells, i.e. cells whose plasma membrane has lost integrity, has been demonstrated in in vitro tests.

The term selectively indicates that the targeting compound has a higher affinity for necrotic cells than for healthy cells (thus the targeting molecules may target necrotic cells). Such can be determined in an in vitro assay as per the examples herewith, or e.g. by flow cytometry; in both methods, co-staining may be used e.g. using commercial live-dead cell staining kits. In simple terms, selective binding in the present invention indicates that for a given population of cells comprising necrosis and healthy cells the number of targeting molecules bound to necrotic cells is at least one order of magnitude higher than the number of targeting molecules bound to healthy cells, typically a few orders of magnitude, and preferably 6 or more orders of magnitude, such as 9 orders.

Non-activated cyanines are cyanines that are non-reactive towards e.g. amines and thiols. Non-activated cyanines cannot significantly (reaction is thermodynamically unfavourable) bind to dead cells (functional groups of molecules thereof) through covalently attaching to amines, thiols or other reactive functional groups present on molecules found inside of cells. That is to say that selective binding to necrotic cells in the context of the present invention is not through covalent bonding, but rather through non-covalent binding via the cynanine core structure and not through the side chains to which the active groups are attached. The term activated cyanine is known to a person of skill in the art and includes e.g. carboxylic acids activated as esters, N-hydroxysuccinimide esters, maleimides, acyl chlorides, SDS esters, etc. Non-activated cyanines include e.g. cyaninines comprising carboxylic acid functions i.e. the carboxylic acid is not activated.

In a further aspect the present invention relates to a use of a dosage according to the invention in an in vivo method for thermal-therapy, the treatment comprising an increase of an internal temperature of the living tumor cells, such as to an internal temperature of at least 42° C.

Advantages of the present description are detailed throughout the description.

### DETAILED DESCRIPTION

It is noted that examples given, as well as embodiments are not considered to be limiting. The scope of the invention is defined by the claims.

In a first aspect the present invention relates a targeting system according to claim 1. Upon testing especially the above cyanines have been found to be very suitable, e.g. in terms of selectivity. Advantageously, cyanines of the invention having a negative charge have been found to bind preferentially to intracellular proteins in the presence of other cell components such as e.g. DNA and RNA. That is to say, cyanines of the invention having a negative charge show in general no significant binding to DNA or RNA. In an exemplary embodiment of the present system the targeting molecule is therefore neutral or negatively charged, wherein neutral cyanines are also found to perform well.

In an exemplary embodiment of the present system the targeting molecule-chelator is selected from HQ4-DOTA, HQ5-DOTA, CW 800-DOTA, ZW800-DOTA, HQ4-NOTA, HQ5-NOTA, CW 800-NOTA, ZW800-NOTA, and combinations thereof. These combinations of cyanine/chelator are found to be of particular interest in clinical trials.

In an exemplary embodiment of the present system the radionuclide is selected from a group consisting of ⁶⁴Cu, ⁶⁷Cu, ⁶⁷Ga, ⁶⁸Ga, ⁷⁰Ga, ⁷²Ga, ⁸⁹Zr, ⁹⁰Y, ⁹⁵Zr, ¹¹¹In, ¹¹⁴In, ¹²³I, ¹²⁴I, ¹⁵³Gd, ¹⁵⁹Gd, and ¹⁷⁷Lu, and combinations thereof, wherein the radionuclide is optionally present as a cation, such as with a valence of 0, 1, 2, 3, or 4, such as Cu⁺, Cu²⁺, Cu³⁺, Cu⁴⁺, Ga⁺, Ga²⁺, Ga³⁺, Gd⁺, Gd²⁺, Gd³⁺, I+, I³⁺, In⁺, In²⁺, In³⁺, Lu³⁺, Zr⁺, Zr²⁺, Zr³⁺, Zr⁴⁺, Y²⁺, and Y³⁺. Especially these radionuclides are found to provide sufficient action within relative short time frames, with limited side-effects.

In an exemplary embodiment of the present system the chelator-radionuclide is selected from ⁶⁴Cu_{y}-DOTA, ⁶⁷Cu_{y}-DOTA, ⁶⁷Ga_{y}-DOTA, ⁶⁸Ga_{y}-DOTA, ⁷⁰Ga_{y}-DOTA, ⁷²Ga_{y}-DOTA, ⁸⁹Zr_{y}-DOTA, ⁹⁰Y_{y}-DOTA, ⁹⁵Zr_{y}-DOTA, ¹¹¹In_{y}-DOTA, ¹¹⁴In_{y}-DOTA, ¹²³I_{y}-DOTA, ¹²⁴I_{y}-DOTA, ¹⁵³Gd_{y}-DOTA, ¹⁵⁹Gd_{y}-DOTA, ¹⁷⁷Lu_{y}-DOTA, ⁶⁴Cu_{y}-NOTA, ⁶⁷Cu_{y}-NOTA, ⁶⁷Ga_{y}-NOTA, ⁶⁸Ga_{y}-NOTA, ⁷⁰Ga_{y}-NOTA, ⁷²Ga_{y}-NOTA, ⁸⁹Zr_{y}-NOTA, ⁹⁰Y_{y}-NOTA, ⁹⁵Zr_{y}-NOTA, ¹¹¹In_{y}-NOTA, ¹¹⁴In_{y}-NOTA, ¹²³I_{y}-NOTA, ¹²⁴I_{y}-NOTA, ¹⁵³Gd_{y}-NOTA, ¹⁵⁹Gd_{y}-NOTA, ⁷⁷Lu_{y}-NOTA, and combinations thereof, wherein y∈[1,2,3,4]. These combinations of radionuclide/chelator are found to be of particular interest in clinical trials.

In an exemplary embodiment of the present system the combination of cyanine and radionuclide is selected from ⁶⁴Cu/HQ4, ⁶⁷Cu/HQ4, ⁶⁷Ga/HQ4, ⁶⁸Ga/HQ4, ⁷⁰Ga/HQ4, ⁷²Ga/HQ4, ⁸⁹Zr/HQ4, ⁹⁰Y/HQ4, ⁹⁵Zr/HQ4, ¹¹¹In/HQ4, ¹¹⁴In/HQ4, ¹²³I/HQ4, ¹²⁴I/HQ4, ¹⁵³Gd/HQ4, ¹⁵⁹Gd/HQ4, ¹⁷⁷Lu/HQ4, ⁶⁴Cu/HQ5, ⁶⁷Cu/HQ5, ⁶⁷Ga/HQ5, ⁶⁸Ga/HQ5, ⁷⁰Ga/HQ5, ⁷²Ga/HQ5, ⁸⁹Zr/HQ5, ⁹⁰Y/HQ5, ⁹⁵Zr/HQ5, ¹¹¹In/HQ5, ¹¹⁴In/HQ5, ¹²³I/HQ5, ¹²⁴I/HQ5, ¹⁵³Gd/HQ5, ¹⁵⁹Gd/HQ5, ¹⁷⁷Lu/HQ5, ⁶⁴Cu/CW800, ⁶⁷Cu/CW800, ⁶⁷Ga/CW800, ⁶⁸Ga/CW800, ⁷⁰Ga/CW800, ⁷²Ga/CW800, ⁸⁹Zr/CW800, ⁹⁰Y/CW800, ⁹⁵Zr/CW800, ¹¹¹In/CW800, ¹¹⁴In/CW800, ¹²³I/CW800, ¹²⁴I/CW800, ¹⁵³Gd/CW800, ¹⁵⁹Gd/CW800, ¹⁷⁷Lu/CW800, ⁶⁴Cu/ZW800, ⁶⁷Cu/ZW800, ⁶⁷Ga/ZW800, ⁶⁸Ga/ZW800, ⁷⁰Ga/ZW800, ⁷²Ga/ZW800, ⁸⁹Zr/ZW800, ⁹⁰Y/ZW800, ⁹⁵Zr/ZW800, ¹¹¹In/ZW800, ¹¹⁴In/ZW800, ¹²³I/ZW800, ¹²⁴I/ZW800, ¹⁵³Gd/ZW800, ¹⁵⁹Gd/ZW800, ¹⁷⁷Lu/ZW800, and combinations thereof.

In an exemplary embodiment the present system for use as a medicament for esophagus cancer is selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ⁷⁷Lu_{y}-NOTA-ZW800, and combinations thereof, wherein y∈[1,2,3,4].

In an exemplary embodiment the present system for use as a medicament for lung cancer is selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, 111In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, 111In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, 111In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

In an exemplary embodiment the present system for use as a medicament for intestine cancer is selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, 111In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, 111In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, 111In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, 111In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ⁷⁷Lu_{y}-NOTA-ZW800, and combinations thereof, wherein y∈[1,2,3,4].

In an exemplary embodiment the present system for use as a medicament for brain cancer is selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, 111In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, 111In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

In an exemplary embodiment the present system for use as a medicament for pharynx or larynx cancer is selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴1y-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴1y-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, 111In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴1y-NOTA-HQ4, ¹⁵³Gd_{y}-NOTA-HQ4, ¹⁵⁹Gd_{y}-NOTA-HQ4, ⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, 111In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, 111In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, 111In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

In a second aspect the present invention relates to a dosage as a drug for treatment of cancers selected from esophagus, pharynx and larynx, lung, brain, and intestines, comprising an effective amount of the system of the invention.

In an example the dosage comprises an amount of 0.1-1000 nMole system/kg body weight, preferably 0.5-500 nMole system/kg body weight, more preferably 1-250 nMole system/kg body weight, even more preferably 2-100 nMole system/kg body weight, such as 5-50 nMole system/kg body weight; such may relate to a dosage of e.g. 0.01-200 mgram. The dosage preferably is provided in a physiological solution of 1-50 ml. Preferably a kit comprising some (1-50) dosages is provided.

The invention is further detailed by the Examples and accompanying figures, which are exemplary and explanatory of nature and are not limiting the scope of the invention. To the person skilled in the art it may be clear that many variants, being obvious or not, may be conceivable falling within the scope of protection, defined by the present claims.

### SUMMARY OF FIGURES

FIG. 1a-g shows cyanines, FIG. 2a-b chelators, and FIG. 3 a complex.

### DETAILS OF THE FIGURES

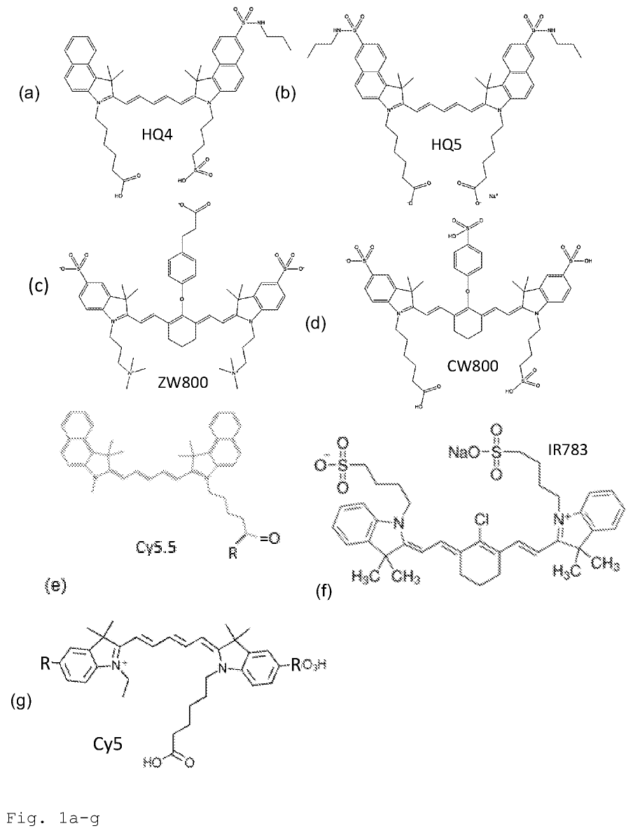

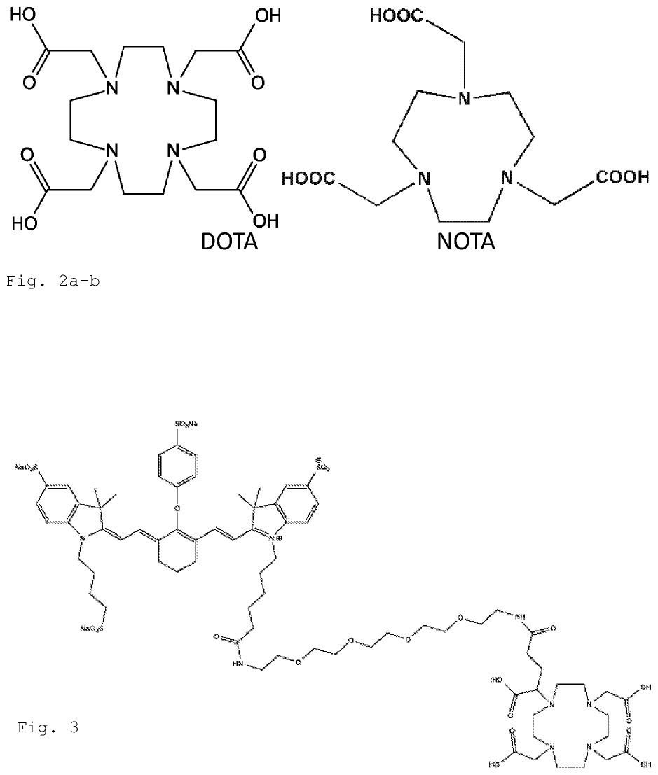

FIG. 1a-g shows chemical structures of various cyanines. FIGS. 1a-d are cyanines according to the invention, FIGS. 1e-1g according to labels of the prior art.

FIG. 2a-b shows chemical structures of DOTA and NOTA.

FIG. 3 shows a cyanine-DOTA complex.

### Examples

Clinical Trials

Various clinical trials are performed. For some cancers, namely lung cancer, esophagus cancer, brain cancer, larynx/pharynx cancer, and cancer of the intestines populations of patients are followed during treatment, therapy and diagnosis.

At the University Hospital Tubingen (UKT) various clinical trials are performed with the present targeting system as both diagnostic and therapeutic medicine for esophagus-, brain-, pharynx- and larynx-, lung-, and intestines-cancer. In this clinical trials cancer patients are followed during protocols of diagnosis, treatment and therapy.

For the diagnostic treatment the chelator is labeled with a radionuclide, like Zirconium, Gallium, Gadolinium, or Copper, and intravenously administered to the patient followed by medical imaging with a PET-, SPECT- or MRI-imaging device. Diagnostic treatment of necro-sis results in the detection, localization and quantify of necrotic tissue in the human body.

For the radio-therapeutic treatment the chelator is labeled with a radionuclide like Copper or Lutetium and administered to the patient. The radionuclide radiates the living tumor-cells from within the tumor, as it is bound to the necrotic tumor cores.

For the Hyperthermia-treatment the chelator is labeled with a magnetic molecule like gadolinium and administered to the patient intravenously. As the gadolinium is bound to the necrotic cores in the tumor, the surrounding living tumor is selectively heated with an MRI-imaging device for increasing the tumor cells to 42° C. or more, which leads to increase of living tumor cells.

### Results

For all trials initial results indicate that the present targeting system increases necrosis at a location of the respective cancers.

In addition it has been found that the present system can be used to establish the effect of a further treatment, such as immunotherapy, chemotherapy, proton therapy, and radiotherapy, by using the targeting system to attach to the necrotic cells formed as a result of the further treatment. Such is important, as now in an early stage an effect of the further treatment can be established; if no effect or a small effect is visible, the further treatment can be stopped.

The present targeting system itself is also effective as radiotherapy, as the radionuclide of the system increases local necrosis, as is intended.

As the present system can be used in very low dosage regimes, no side-effects are observed. In addition it has been confirmed that the present system can be cleared form the human body to a high extent.

It is noted that a healthy human body does not contain necrotic tissue, which means that when necrosis is detected by the present targeting system this indicates a pathological situation like stroke, myocardial infarction, osteo-arthritis and/or a aggressively growing tumor. Aggressively growing (malign) tumors and/or metastases develop a necrotic core starting at a diameter of 3 mm or more. Detecting necrosis in a human body is relevant because based on this outcome medical professionals can diagnose medical issues, determine treatment options and make medical relevant decisions of which the patient can benefit.

In addition it has been found that the present system can be used to establish the effect of a further treatment, such as immunotherapy, chemotherapy, by using the targeting system to attach to the necrotic cells formed as a result of the further treatment. Such is important, as now in an early stage an effect of the further treatment can be established; if no effect or a small effect is visible, the further treatment can be stopped.

The present targeting system provides a new early stage method to determine the efficacy of the treatment of aggressively growing (malign) tumors. The outcome and results of conventional therapies like chemotherapy and radio-therapy, but also new therapies like immune-, brachy- or proton-therapy is only available after multiple treatments. Moreover, the outcome of chemo- and immunotherapy is only known after multiple treatments over a longer period of time.

Today shrinkage and decrease of volume of the tumor are indications that a treatment is effective. Fact is that in average chemotherapies are costly but only effective in a minority of the cases. With the present targeting system necrosis in the tumor is quantified before (T=0) and shortly after (T=1) the administration of the chemo- or immunotherapy. The delta of the radioactive signal between T=0 and T=1 reflects the efficacy of the administered therapy. Implementing this application with the present targeting system provides a reduction of the ineffective treatments with 35% or more. Besides improving quality of life by avoiding severe treatments of patients which do not have to suffer from a treatment which does not provide the desired effect, the cost reduction of cancer treatment is tremendous. Cancer treatment (direct and side-effect treatment) represent 75% or more of the total budget for drugs and treatments in an average hospital. Possible reductions in costs of care and medicines in the Western world alone represent at least € 2 Billions.

The present targeting system itself is also effective as radiotherapy, as the radionuclide of the system increases local necrosis by killing living tumor-cells with nuclear radiation, as is intended. It is found that typically a volume of tumor-cells is reduced by 50% or more, and often these are not visible anymore. In addition the present treatment provides sufficient control of the tumor, and limits further development thereof, such that patients require on regular treatment at the most; hence patients can now be treated comparable to other long-term diseases and continue mostly or fully with their lives.

As the present system can be used in very low dosage regimes, the treatment is targeted and limited to the tumor and no side-effects are observed. In addition it has been confirmed that the present system binds to the necrotic core of tumors and the unbound compounds can be cleared from the human body to a high extent within a maximum of 24-hours after administration. This new hyperthermia treatment options provides important new treatment options for non-resectable or non-treatable aggressively growing (malign) tumors. This new targeted in-tumor radiotherapy treatment provides important new options for non-resectable or non-treatable aggressively growing (malign) tumors, such as the ones claimed.

The present targeting system itself is also effective as thermal-therapy, the treatment of the iron-labelled chelator with MRI results in the increase of the internal temperature of the living tumor cells. After the internal temperature of these cells is 42 □C, or higher, this treatment results in the death of this tumor cells. This new form of hyperthermia treatment provides important new treatment options for non-receptable or non-treatable aggressively growing (malign) tumors.

Study results show an increase of dead (effectively treat-ed) tumor in 40-60% of tumors (as is 35% on average for to-day's chemotherapy). Repetitive treatments with present targeting system for the same tumor show increase of tumor death by 40-60% for each treatment-cycle that can add up to eradicating all of living cells in a tumor.

1. A targeting system for use as a drug for cancers selected from esophagus, pharynx and larynx, lung, brain, and intestines, the system comprising a targeting molecule for binding to necrotic cells, the targeting molecule being a cyanine selected from HQ4, HQ5, CW-800 and ZW-800, and combinations thereof, 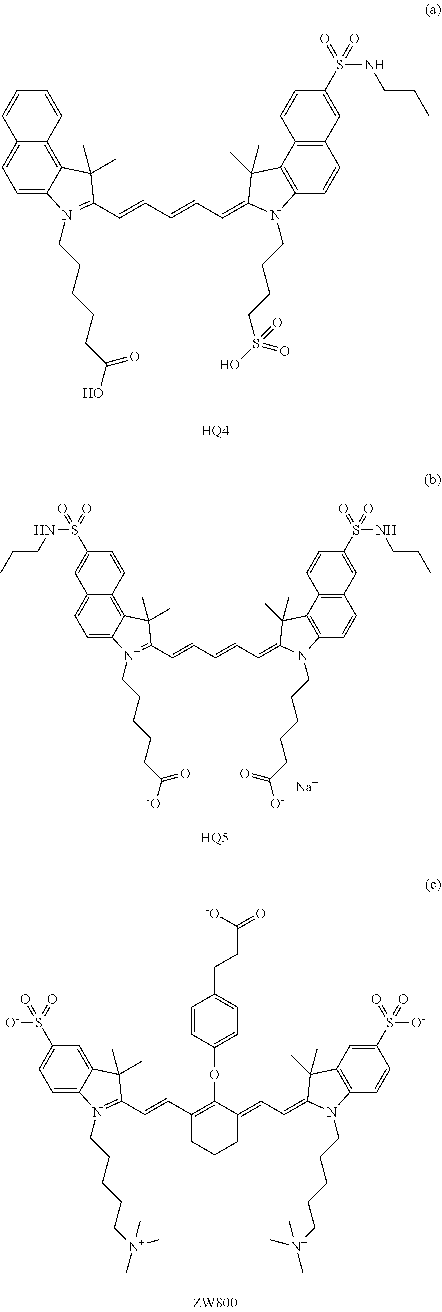 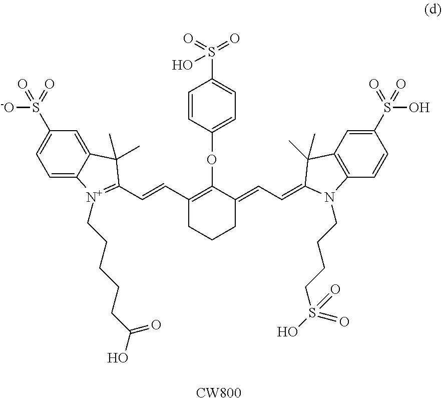
wherein the cyanine is attached to a chelator, wherein the chelator is selected from DOTA and NOTA, and combinations thereof, 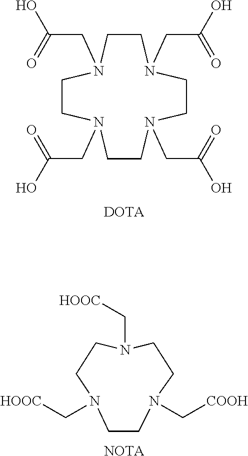
and attached to the chelator a radionuclide selected from Cu, In, Gd, Ga, Lu, Y, I, and Zr, and combinations thereof.

2. The targeting system according to claim 1 wherein the targeting molecule is neutral or negatively charged.

3. The targeting system according to claim 1, wherein the targeting molecule-chelator is selected from HQ4-DOTA, HQ5-DOTA, CW 800-DOTA, ZW800-DOTA, HQ4-NOTA, HQ5-NOTA, CW 800-NOTA, ZW800-NOTA, and combinations thereof.

4. The targeting system according to claim 1, wherein the radionuclide is selected from a group consisting of ⁶⁴Cu, ⁶⁷Cu, ⁶⁷Ga, ⁶⁸Ga, ⁷⁰Ga, ⁷²Ga, ⁸⁹Zr, ⁹⁰Y, ⁹⁵Z, ¹¹¹In, ¹¹⁴In, ¹²³I, ¹²⁴I, ¹⁵³Gd, ¹⁵⁹Gd, and ¹⁷⁷Lu, and combinations thereof, wherein the radionuclide is present as a cation, with a valence of 0, 1, 2, 3, or 4, comprising at least one of Cu⁺, Cu²⁺, Cu³⁺, Cu⁴⁺, Ga⁺, Ga²⁺, Ga³⁺, Gd⁺, Gd²⁺, Gd³⁺, I⁺, I³⁺, In⁺, In²⁺, In³⁺, Lu³⁺, Zr, Zr²⁺, Zr³⁺, Zr⁴⁺, Y²⁺, and Y³⁺.

5. The targeting system according to claim 1, wherein the chelator-radionuclide is selected from ⁶⁴Cu_{y}-DOTA, ⁶⁷Cu_{y}-DOTA, ⁶⁷Ga_{y}-DOTA, ⁶⁸Ga_{y}-DOTA, ⁷⁰Ga_{y}-DOTA, ⁷²Ga_{y}-DOTA, ⁸⁹Zr_{y}-DOTA, ⁹⁰Y_{y}-DOTA, ⁹⁵Zr_{y}-DOTA, ¹¹¹In_{y}-DOTA, ¹¹⁴In_{y}-DOTA, ¹²³I_{y}-DOTA, ¹²⁴I_{y}-DOTA, ¹⁵³Gd_{y}-DOTA, ¹⁵⁹Gd_{y}-DOTA, ¹⁷⁷Lu_{y}-DOTA, ⁶⁴Cu_{y}-NOTA, ⁶⁷Cu_{y}-NOTA, ⁶⁷Ga_{y}-NOTA, ⁶⁸Ga_{y}-NOTA, ⁷⁰Ga_{y}-NOTA, ⁷²Ga_{y}-NOTA, ⁸⁹Zr_{y}-NOTA, ⁹⁰Y_{y}-NOTA, ⁹⁵Zr_{y}-NOTA, ¹¹¹In_{y}-NOTA, ¹¹⁴In_{y}-NOTA, ¹²³I_{y}-NOTA, ¹²⁴I_{y}-NOTA, ¹⁵³Gd_{y}-NOTA, ¹⁵⁹Gd_{y}-NOTA, ¹⁷⁷Lu_{y}-NOTA, and combinations thereof, wherein y∈[1,2,3,4].

6. The targeting system according to claim 1, wherein the combination of cyanine and radionuclide is selected from ⁶⁴Cu/HQ4, ⁶⁷Cu/HQ4, ⁶⁷Ga/HQ4, ⁶⁸Ga/HQ4, ⁷⁰Ga/HQ4, ⁷²Ga/HQ4, ⁸⁹Zr/HQ4, ⁹⁰Y/HQ4, ⁹⁵Zr/HQ4, ¹¹¹In/HQ4, ¹¹⁴In/HQ4, ¹²³/HQ4, ¹²⁴I/HQ4, ¹⁵³Gd/HQ4, ¹⁵⁹Gd/HQ4, ¹⁷⁷Lu/HQ4, ⁶⁴Cu/HQ5, ⁶⁷Cu/HQ5, ⁶⁷Ga/HQ5, ⁶⁸Ga/HQ5, ⁷⁰Ga/HQ5, ⁷²Ga/HQ5, ⁸⁹Zr/HQ5, ⁹⁰Y/HQ5, ⁹⁵Zr/HQ5, ¹¹¹In/HQ5, ¹¹⁴In/HQ5, ¹²³I/HQ5, ¹²⁴I/HQ5, ¹⁵³Gd/HQ5, ¹⁵⁹Gd/HQ5, ¹⁷⁷Lu/HQ5, ⁶⁴Cu/CW800, ⁶⁷Cu/CW800, ⁶⁷Ga/CW800, ⁶⁸Ga/CW800, ⁷⁰Ga/CW800, ⁷²Ga/CW800, ⁸⁹Zr/CW800, ⁹⁰Y/CW800, ⁹⁵Zr/CW800, ¹¹¹In/CW800, ¹¹⁴In/CW800, ¹²³I/CW800, ¹²⁴I/CW800, ¹⁵³Gd/CW800, ¹⁵⁹Gd/CW800, ¹⁷⁷Lu/CW800, ⁶⁴Cu/ZW800, ⁶⁷Cu/ZW800, ⁶⁷Ga/ZW800, ⁶⁸Ga/ZW800, ⁷⁰Ga/ZW800, ⁷²Ga/ZW800, ⁸⁹Zr/ZW800, ⁹⁰Y/ZW800, ⁹⁵Zr/ZW800, ¹¹¹In/ZW800, ¹¹⁴In/ZW800, ¹²³I/ZW800, ¹²⁴I/ZW800, ¹⁵³Gd/ZW800, ¹⁵⁹Gd/ZW800, ¹⁷⁷Lu/ZW800, and combinations thereof.

7. A targeting system drug for esophagus cancer selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein ye[1,2,3,4].

8. A targeting system drug for lung cancer selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

9. A targeting system drug for intestine cancer selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

10. A targeting system drug for brain cancer selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

11. A targeting system drug for pharynx cancer or for larynx cancer selected from ⁶⁴Cu_{y}-DOTA-HQ4, ⁶⁷Cu_{y}-DOTA-HQ4, ⁶⁷Ga_{y}-DOTA-HQ4, ⁶⁸Ga_{y}-DOTA-HQ4, ⁷⁰Ga_{y}-DOTA-HQ4, ⁷²Ga_{y}-DOTA-HQ4, ⁸⁹Zr_{y}-DOTA-HQ4, ⁹⁰Y_{y}-DOTA-HQ4, ⁹⁵Zr_{y}-DOTA-HQ4, ¹¹¹In_{y}-DOTA-HQ4, ¹¹⁴In_{y}-DOTA-HQ4, ¹²³I_{y}-DOTA-HQ4, ¹²⁴I_{y}-DOTA-HQ4, ¹⁵³Gd_{y}-DOTA-HQ4, ¹⁵⁹Gd_{y}-DOTA-HQ4, ¹⁷⁷Lu_{y}-DOTA-HQ4, ⁶⁴Cu_{y}-DOTA-HQ5, ⁶⁷Cu_{y}-DOTA-HQ5, ⁶⁷Ga_{y}-DOTA-HQ5, ⁶⁸Ga_{y}-DOTA-HQ5, ⁷⁰Ga_{y}-DOTA-HQ5, ⁷²Ga_{y}-DOTA-HQ5, ⁸⁹Zr_{y}-DOTA-HQ5, ⁹⁰Y_{y}-DOTA-HQ5, ⁹⁵Zr_{y}-DOTA-HQ5, ¹¹¹In_{y}-DOTA-HQ5, ¹¹⁴In_{y}-DOTA-HQ5, ¹²³I_{y}-DOTA-HQ5, ¹²⁴I_{y}-DOTA-HQ5, ¹⁵³Gd_{y}-DOTA-HQ5, ¹⁵⁹Gd_{y}-DOTA-HQ5, ¹⁷⁷Lu_{y}-DOTA-HQ5, ⁶⁴Cu_{y}-DOTA-CW800, ⁶⁷Cu_{y}-DOTA-CW800, ⁶⁷Ga_{y}-DOTA-CW800, ⁶⁸Ga_{y}-DOTA-CW800, ⁷⁰Ga_{y}-DOTA-CW800, ⁷²Ga_{y}-DOTA-CW800, ⁸⁹Zr_{y}-DOTA-CW800, ⁹⁰Y_{y}-DOTA-CW800, ⁹⁵Zr_{y}-DOTA-CW800, ¹¹¹In_{y}-DOTA-CW800, ¹¹⁴In_{y}-DOTA-CW800, ¹²³I_{y}-DOTA-CW800, ¹²⁴I_{y}-DOTA-CW800, ¹⁵³Gd_{y}-DOTA-CW800, ¹⁵⁹Gd_{y}-DOTA-CW800, ¹⁷⁷Lu_{y}-DOTA-CW800, ⁶⁴Cu_{y}-DOTA-ZW800, ⁶⁷Cu_{y}-DOTA-ZW800, ⁶⁷Ga_{y}-DOTA-ZW800, ⁶⁸Ga_{y}-DOTA-ZW800, ⁷⁰Ga_{y}-DOTA-ZW800, ⁷²Ga_{y}-DOTA-ZW800, ⁸⁹Zr_{y}-DOTA-ZW800, ⁹⁰Y_{y}-DOTA-ZW800, ⁹⁵Zr_{y}-DOTA-ZW800, ¹¹¹In_{y}-DOTA-ZW800, ¹¹⁴In_{y}-DOTA-ZW800, ¹²³I_{y}-DOTA-ZW800, ¹²⁴I_{y}-DOTA-ZW800, ¹⁵³Gd_{y}-DOTA-ZW800, ¹⁵⁹Gd_{y}-DOTA-ZW800, ¹⁷⁷Lu_{y}-DOTA-ZW800, ⁶⁴Cu_{y}-NOTA-HQ4, ⁶⁷Cu_{y}-NOTA-HQ4, ⁶⁷Ga_{y}-NOTA-HQ4, ⁶⁸Ga_{y}-NOTA-HQ4, ⁷⁰Ga_{y}-NOTA-HQ4, ⁷²Ga_{y}-NOTA-HQ4, ⁸⁹Zr_{y}-NOTA-HQ4, ⁹⁰Y_{y}-NOTA-HQ4, ⁹⁵Zr_{y}-NOTA-HQ4, ¹¹¹In_{y}-NOTA-HQ4, ¹¹⁴In_{y}-NOTA-HQ4, ¹²³I_{y}-NOTA-HQ4, ¹²⁴I_{y}-NOTA-HQ4, ¹⁵³Gd_{y}-NOTA-HQ4, ¹⁵⁹Gd_{y}-NOTA-HQ4, ¹⁷⁷Lu_{y}-NOTA-HQ4, ⁶⁴Cu_{y}-NOTA-HQ5, ⁶⁷Cu_{y}-NOTA-HQ5, ⁶⁷Ga_{y}-NOTA-HQ5, ⁶⁸Ga_{y}-NOTA-HQ5, ⁷⁰Ga_{y}-NOTA-HQ5, ⁷²Ga_{y}-NOTA-HQ5, ⁸⁹Zr_{y}-NOTA-HQ5, ⁹⁰Y_{y}-NOTA-HQ5, ⁹⁵Zr_{y}-NOTA-HQ5, ¹¹¹In_{y}-NOTA-HQ5, ¹¹⁴In_{y}-NOTA-HQ5, ¹²³I_{y}-NOTA-HQ5, ¹²⁴I_{y}-NOTA-HQ5, ¹⁵³Gd_{y}-NOTA-HQ5, ¹⁵⁹Gd_{y}-NOTA-HQ5, ¹⁷⁷Lu_{y}-NOTA-HQ5, ⁶⁴Cu_{y}-NOTA-CW800, ⁶⁷Cu_{y}-NOTA-CW800, ⁶⁷Ga_{y}-NOTA-CW800, ⁶⁸Ga_{y}-NOTA-CW800, ⁷⁰Ga_{y}-NOTA-CW800, ⁷²Ga_{y}-NOTA-CW800, ⁸⁹Zr_{y}-NOTA-CW800, ⁹⁰Y_{y}-NOTA-CW800, ⁹⁵Zr_{y}-NOTA-CW800, ¹¹¹In_{y}-NOTA-CW800, ¹¹⁴In_{y}-NOTA-CW800, ¹²³I_{y}-NOTA-CW800, ¹²⁴I_{y}-NOTA-CW800, ¹⁵³Gd_{y}-NOTA-CW800, ¹⁵⁹Gd_{y}-NOTA-CW800, ¹⁷⁷Lu_{y}-NOTA-CW800, ⁶⁴Cu_{y}-NOTA-ZW800, ⁶⁷Cu_{y}-NOTA-ZW800, ⁶⁷Ga_{y}-NOTA-ZW800, ⁶⁸Ga_{y}-NOTA-ZW800, ⁷⁰Ga_{y}-NOTA-ZW800, ⁷²Ga_{y}-NOTA-ZW800, ⁸⁹Zr_{y}-NOTA-ZW800, ⁹⁰Y_{y}-NOTA-ZW800, ⁹⁵Zr_{y}-NOTA-ZW800, ¹¹¹In_{y}-NOTA-ZW800, ¹¹⁴In_{y}-NOTA-ZW800, ¹²³I_{y}-NOTA-ZW800, ¹²⁴I_{y}-NOTA-ZW800, ¹⁵³Gd_{y}-NOTA-ZW800, ¹⁵⁹Gd_{y}-NOTA-ZW800, ¹⁷⁷Lu_{y}-NOTA-ZW800, and combinations thereof, wherein y∈[1,2,3,4].

12. A drug dosage for treatment of cancers selected from esophagus, pharynx and larynx, lung, brain, and intestines, comprising an effective amount of the system of claim 1.

13. (canceled)

14. The drug dosage according to claim 12, comprising an amount of 0.1-1000 nMole system/kg body weight and provided in a physiological solution of 1-50 ml.

15. The drug dosage according to claim 14 for thermal-therapy, the therapy comprising an increase of an internal temperature of the living tumor cells, to an internal temperature of at least 42° C.
